(19) United States
(12) Patent Application Publication
(10) Pub. No.: US 2017/0151151 A1
(43) Pub. Date: Jun. 1, 2017

(54) ANTIPERSPIRANT COSMETICS COMPRISING PROTEINS DERIVED FROM MALVACEAE OF THE SPECIES ANDANSONIA WHICH ARE EXEMPT OF ALUMINUM AND/OR ZIRCONIUM HALIDES AND/OR HYDROXY HALIDES

(71) Applicant: Henkel AG & Co. KGaA, Duesseldorf (DE)

(72) Inventors: Bernhard Banowski, Duesseldorf (DE); Stefan Evers, Haan (DE)

(73) Assignee: Henkel AG & Co. KGaA, Duesseldorf (DE)

(21) Appl. No.: 15/430,187

(22) Filed: Feb. 10, 2017

Related U.S. Application Data
(63) Continuation of application No. PCT/EP2015/064557, filed on Jun. 26, 2015.

(30) Foreign Application Priority Data
Aug. 26, 2014 (DE) 10 2014 216 913.5

(51) Int. Cl. A61K 8/64; A61K 8/04; A45D 34/00; B65D 83/14; B65D 25/02; A61Q 15/00; A61K 8/19

(52) U.S. Cl. CPC A61K 8/64; A61Q 15/00; A61K 8/046; A61K 8/19; B65D 83/752; B65D 25/02; A45D 34/00; A61K 2800/30; A61K 2800/88; A61K 2800/87; A61K 2800/524

(57) ABSTRACT

The present invention relates to an antiperspirant cosmetic agent that includes at least one specific protein from Malvaceae of the genus Adansonia and is free of aluminum and/or zirconium halides and/or hydroxy halides. The present invention further relates to the use of a specific protein and to a non-therapeutic method for reducing body perspiration. Adding or using said at least one specific protein ensures that the sweat gland(s) is/are effectively influenced, thus resulting in a significant reduction in underarm sweat even in the absence of antiperspirant aluminum salts.

### FIELD OF THE INVENTION

The present invention generally relates to an antiperspirant cosmetic including at least one substance selected from the group consisting of cosmetic oils that are liquid at 20° C. and 1,013 hPa, odorants, and waxes, optionally at least one propellant, and specific proteins made from Malvaceae of the genus Adansonia. The cosmetic agents according to the present invention include no antiperspirant aluminum and/or zirconium halides and/or hydroxy halides. Adding the at least one specific protein results in an influence on the sweat gland(s).

The present invention further relates to a packaging unit (kit-of-parts) including a cosmetic agent according to the present invention, as well as a cosmetic agent having at least one antiperspirant active ingredient.

The present invention additionally relates to the use of specific proteins made from Malvaceae of the genus Adansonia to at least partially influence sweat gland(s).

The present invention moreover relates to the use of a combination including at least one substance selected from the group consisting of cosmetic oils that are liquid at 20° and 1,013 hPa, odorants, and waxes, optionally at least one propellant, and specific proteins made from Malvaceae of the genus Adansonia, to reduce and/or prevent sweat, in particular, underarm sweat or sweat from other regions of the body. The combination according to the present invention includes no antiperspirant aluminum and/or zirconium halides and/or hydroxy halides.

The present invention also relates to an antiperspirant cosmetic agent that is without aluminum and/or zirconium halides and/or hydroxy halides and includes at least one substance selected from the group consisting of cosmetic oils that are liquid at 20° C. and 1,013 hPa, odorants, and waxes, optionally at least one propellant, and at least one hydrolyzed protein isolated from Malvaceae of the genus Adansonia, preferably a protein isolated and hydrolyzed from the seeds of the genus Adansonia. Adding such proteins results in an influence on the sweat gland(s).

Finally, the present invention relates to a non-therapeutic cosmetic method for preventing and/or reducing body perspiration with which an antiperspirant cosmetic agent according to the present invention or a packaging unit according to the present invention is applied onto the skin, in particular, the skin of the armpits, and remains on the skin of the armpits for at least 1 hour, preferably for at least 2 hours, preferably for at least 4 hours, in particular, for at least 6 hours.

### BACKGROUND OF THE INVENTION

Washing, cleansing, and hygiene of one's own body represent a basic human need, and modern industry constantly attempts to meet these human needs in many ways. The constant elimination or at least reduction of body odor and underarm wetness is particularly important for daily hygiene. In the prior art, numerous specific deodorant or antiperspirant body care products are known which were developed for application in body regions with a high density of sweat glands, in particular, in the armpit region. These are prepared in the most varied forms of application, for example, as a powder, in the form of a stick, as an aerosol spray, pump spray, liquid and gelatinous roll-on application, cream, gel, and as an impregnated flexible substrate (deodorant wipes).

In addition to at least one oil or wax and an odorant component or perfume, cosmetic antiperspirants of the prior art include at least one antiperspirant compound, in particular, in the form of aluminum and/or zirconium halides and/or hydroxy halides. On one hand, these antiperspirant compounds reduce the secretion of sweat from the body by temporarily narrowing and/or blocking the ducts of the sweat glands so that the amount of sweat can be reduced by about 20 to 60%. On the other hand, these antiperspirant compounds have an additional deodorizing effect due to the antimicrobial action thereof.

Aluminum and/or zirconium halides and/or hydroxy halides may lead to unpleasant skin reactions for some users, in connection with the acidic pH of these antiperspirants. Use of the aforementioned antiperspirant compounds can, in addition, lead to formation of spots on clothing.

There is therefore a need to replace antiperspirant aluminum and/or zirconium halides and/or hydroxy halides with other antiperspirant cosmetic active ingredients. The antiperspirant active ingredients should have a favorable antiperspirant action and favorable skin compatibility, and be easily formulated. These antiperspirant active ingredients should also have no negative impact on the storage stability of the antiperspirant cosmetic agents.

The present invention addresses the problem of providing an antiperspirant cosmetic agent that avoids or at least mitigates the disadvantages of the prior art and that has favorable skin compatibility while simultaneously reliably reducing underarm wetness. The antiperspirant cosmetic agents should moreover have a long shelf life. It has now surprisingly been found that using at least one protein from Malvaceae of the genus Adansonia in cosmetic agents results in an antiperspirant effect that is approximately comparable to the antiperspirant effect of formulations comprising aluminum salts and/or aluminum zirconium complexes. The at least one protein can then bring about a change in the light absorption from 1 to 100% when the pH value is changed by at least 0.5 in a pH range of pH 4.0 to pH 8.0. The antiperspirant effect of the agents according to the present invention is achieved without the addition of antiperspirant aluminum and/or zirconium halides and/or hydroxy halides.

Furthermore, other desirable features and characteristics of the present invention will become apparent from the subsequent detailed description of the invention and the appended claims, taken in conjunction with this background of the invention.

### BRIEF SUMMARY OF THE INVENTION

An antiperspirant cosmetic agent, including: at least one substance selected from the group consisting of cosmetic oils that are liquid at 20° C. and 1,013 hPa, odorants, and waxes; propellants in a total amount of 0 to 99 wt %, relative to the total weight of the antiperspirant cosmetic agent; and at least one protein in a total amount of 0.1 to 70 wt % relative to the total weight of the antiperspirant cosmetic agent, wherein the at least one protein is found in Malvaceae of the genus Adansonia, and wherein the at least one protein brings about a change in the light absorption of 1 to 100% when there is a change in pH value of at least 0.5 in a pH range of pH 4.0 to pH 8.0, a temperature of 20° C. to 40°, and a concentration of the protein of 0.001 to 10 wt % relative to the total weight of the sample mixture used to determine the change in the light absorption; wherein the antiperspirant cosmetic agents include no aluminum and/or zirconium halides and/or hydroxy halides.

A packaging unit (kit-of-parts) comprising—prepared separately from one another—at least one first container (C1) containing a cosmetic agent (M1) comprising at least one antiperspirant active ingredient, and at least one second container (C2) containing a cosmetic agent (M2) including at least one protein, wherein the at least one protein is found in Malvaceae of the genus Adansonia, wherein the at least one protein brings about a change in the light absorption of 1 to 100% when there is a change in pH value of at least 0.5 in a pH range of pH 4.0 to pH 8.0, a temperature of 20° C. to 40°, and a concentration of the protein of 0.001 to 10 wt % relative to the total weight of the sample mixture used to determine the change in the light absorption, and wherein the cosmetic agent (M2) includes no aluminum and/or zirconium halides and/or hydroxy halides.

Use of at least one protein to at least partially influence the sweat gland(s), wherein the at least one protein is found in Malvaceae of the genus Adansonia, and wherein the at least one protein brings about a change in the light absorption of 1 to 100% when there is a change in pH value of at least 0.5 in a pH range of pH 4.0 to pH 8.0, a temperature of 20° C. to 40°, and a concentration of the protein of 0.001 to 10 wt % relative to the total weight of the sample mixture used to determine the change in the light absorption.

Use of a combination, including at least one substance selected from the group consisting of cosmetic oils that are liquid at 20° C. and 1,013 hPa, odorants, and waxes; propellants in a total amount of 0 to 99 wt %, relative to the total weight of the antiperspirant cosmetic agent; and at least one protein in a total amount of 0.1 to 70 wt % relative to the total weight of the antiperspirant cosmetic agent, wherein the at least one protein is found in Malvaceae of the genus Adansonia, wherein the at least one protein brings about a change in the light absorption of 1 to 100% when there is a change in pH value of at least 0.5 in a pH range of pH 4.0 to pH 8.0, a temperature of 20° C. to 40°, and a concentration of the protein of 0.001 to 10 wt % relative to the total weight of the sample mixture used to determine the change in the light absorption; and wherein the combination includes no aluminum and/or zirconium halides and/or hydroxy halides, to reduce and/or prevent sweat, in particular, underarm sweat or sweat from other regions of the body.

An antiperspirant cosmetic agent, including: at least one substance selected from the group consisting of cosmetic oils that are liquid at 20° C. and 1,013 hPa, odorants, and waxes; propellants in a total amount of 0 to 99 wt %, relative to the total weight of the antiperspirant cosmetic agent; and at least one protein isolated from Malvaceae of the genus Adansonia, preferably a protein isolated from the seeds of the genus Adansonia, in a total amount of 0.1 to 70 wt % relative to the total weight of the antiperspirant cosmetic agent, wherein the protein is hydrolyzed, wherein the antiperspirant cosmetic agent includes no aluminum and/or zirconium halides and/or hydroxy halides.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The subject matter of the present invention is thus an antiperspirant cosmetic agent including:
a) at least one substance selected from the group consisting of cosmetic oils that are liquid at 20° C. and 1,013 hPa, odorants, and waxes;
b) propellants in a total amount of 0 to 99 wt %, relative to the total weight of the antiperspirant cosmetic agent; and
c) at least one protein in a total amount of 0.1 to 70 wt % relative to the total weight of the antiperspirant cosmetic agent, wherein the at least one protein is found in Malvaceae of the genus Adansonia, and wherein the at least one protein brings about a change in the light absorption of 1 to 100% when there is a change in pH value of at least 0.5 in a pH range of pH 4.0 to pH 8.0, a temperature of 20° C. to 40°, and a concentration of the protein of 0.001 to 10 wt % relative to the total weight of the sample mixture used to determine the change in the light absorption;
   wherein the antiperspirant cosmetic agents include no aluminum and/or zirconium halides and/or hydroxy halides.

Using the at least one protein from Malvaceae of the genus Adansonia with the aforementioned specific physical properties in the antiperspirant cosmetic agents according to the present invention results in a targeted influencing of the sweat gland(s)—though no limitation to this theory is intended. This targeted influencing of the sweat gland(s) may, for example, entail gel formation of the at least one protein at pH values that occur exclusively within the ducts of the sweat glands. Effective blockage of the ducts of the sweat glands can thus be ensured without the antiperspirant action of the cosmetic agent according to the present invention being diminished by premature unwanted gel formation due to the addition of the at least one specific protein. The targeted influencing of the sweat gland(s) may also, however, entail disrupting the charge balance within the sweat gland(s), leading to an influence on sweat production, in particular, to a reduction of sweat production Thus, effective reduction of underarm sweat is ensured even in the absence of antiperspirant aluminum and/or zirconium halides and/or hydroxy halides.

According to the present invention, the term “antiperspirant” is understood to mean the decrease or reduction in the perspiration of the sweat glands of the body.

The term “aluminum and/or zirconium halides and/or hydroxy halides” within the framework of the present invention is understood to mean, in particular, chlorides, bromides, and iodides of aluminum and zirconium, as well as compounds of the formulae AI(OH)_{y}X and Zr(OH)_{z}X, where X designates a halide ion in the aforementioned formulae.

In addition, within the meaning of the present invention, the term “cosmetic oil” is understood to mean an oil which is suitable for cosmetic use and which is not miscible with water in all amounts. Moreover, the cosmetic oil used according to the present invention involves neither odorants nor essential oils.

In addition, within the meaning of the present invention, the term “odorants” is understood to mean substances having a molar mass of 74 to 300 g/mol which include at least one osmophoric group in the molecule and which have an odor and/or taste, i.e., which are capable of stimulating the receptors of the hair cells of the olfactory system. Osmophoric groups, in the form of hydroxy groups, formyl groups, oxo groups, alkoxycarbonyl groups, nitrile groups, nitro groups, azide groups, etc., are groups which are covalently bonded to the molecular structure. In this regard, within the meaning of the present invention, perfume oils, perfumes, or perfume oil components that are liquid at 20° C. and 1013 hPa also fall under the term “odorants.”

In addition, the term “wax” within the framework of the present invention is understood to mean substances that are kneadable or solid to brittle at 20° C., have a coarse to finely crystalline structure, and visually are translucent to opaque but not glassy. Moreover, these substances melt above 25° C. without decomposing, are slightly liquid (slightly viscous) at slightly above the melting point, have a strongly temperature-dependent consistency and solubility, and can be polished under slight pressure.

The term “protein” refers according to the present invention to chemical compounds that form condensation products of amino acids amide-linked by peptide bonds. The number of amino acids in the proteins is preferably 2 to 1,000, preferably 2 to 500, in particular, 2 to 60 amino acids. The term “protein” according to the present invention is also understood to mean hydrolysates of proteins including protein fractions with different amino acid sequences and molecular weights. This term is also understood within the framework of the present invention to mean mixtures of proteins found in Malvaceae of the genus Adansonia.

The term “Malvaceae” within the framework of the present invention is understood to mean plants from the Malvales, an order of flowering plants. The best-known crops from the family of the Malvaceae include, for example, the cacao tree, the cotton plant, and the baobab tree.

The term “change in the light absorption of the at least one protein” is understood to mean both positive and negative changes in the light transmittance of the sample mixture, in particular, the protein solution, as well as the absorption of light through the at least one protein or the sample mixture.

The term “change in pH value” is understood to mean the continuous changing of the pH value. The continuous changing of the pH value may be achieved, for example, by titration or uniform addition of a base or acid.

The term “sample mixture” refers according to the present invention to a mixture made of the at least one specific protein with a solvent—in particular, water—buffer, or salt-containing aqueous solutions.

Moreover, the term “fatty acid” as used within the framework of the present invention is understood to mean aliphatic carboxylic acids which have unbranched or branched hydrocarbon moieties including 4 to 40 carbon atoms. The fatty acids used within the framework of the present invention may be naturally occurring as well as synthetically produced fatty acids. In addition, the fatty acids may be monounsaturated or polyunsaturated.

Lastly, within the framework of the present invention, the term “fatty alcohol” is understood to mean aliphatic, monohydric primary alcohols which have unbranched or branched hydrocarbon moieties including 4 to 40 carbon atoms. The fatty alcohols used within the framework of the present invention may also be monounsaturated or polyunsaturated.

Values indicated by wt % presently refer—unless otherwise specified—to the total weight of the antiperspirant cosmetic agents according to the present invention.

As a first component a), the cosmetic agents according to the present invention include at least one substance selected from the group consisting of cosmetic oils that are liquid at 20° C. and 1,013 hPa, odorants, and waxes.

Within the framework of the present invention, the cosmetic oil that is liquid at 20° C. and 1,013 hPa is selected from the group consisting of (i) volatile cyclic silicone oils, in particular, cyclic and linear silicone oils; (ii) volatile non-silicone oils, in particular, liquid paraffin oils and isoparaffin oils; (iii) non-volatile silicone oils; (iv) non-volatile non-silicone oils; and (v) mixtures thereof. The term “volatile oil” refers according to the present invention to oils that—at 20° C. and an ambient pressure of 1,013 hPa—have a vapor pressure of 2.66 Pa to 40,000 Pa (0.02 to 300 mmHg), preferably 10 to 12,000 Pa (0.1 to 90 mmHg), further preferably 13 to 3,000 Pa (0.1 to 23 mmHg), in particular, 15 to 500 Pa (0.1 to 4 mmHg).

Furthermore, within the meaning of the present invention, the term “non-volatile oil” is understood to mean oils having a vapor pressure of less than 2.66 Pa (0.02 mmHg) at 20° C. and an ambient pressure of 1013 hPa.

It may be preferable according to the present invention to use mixtures of volatile silicone oils and volatile non-silicone oils in the antiperspirant cosmetic agents according to the present invention, because so doing achieves a drier skin feel. It may moreover be preferable within the framework of the present invention when the antiperspirant cosmetic agents include a non-volatile silicone oil and/or a non-volatile non-silicone oil in order to mask insoluble components such as talcum or ingredients that have dried onto the skin.

It is especially preferable according to the present invention to use mixtures of non-volatile and volatile cosmetic oils, because so doing makes it possible to adjust parameters such as skin feel, visibility of the residue, and stability of the antiperspirant cosmetic agent according to the present invention, as well as to thus better adapt the agent to the needs of consumers.

The volatile and nonvolatile silicone oils and volatile and non-volatile non-silicone oils that can be used within the framework of the present invention are, for example, disclosed in documents DE 10 2010 063 250 A1 and DE 10 2012 222 692 A1.

According to a preferred embodiment of the present invention, the cosmetic oil that is liquid at 20° C. and 1,013 hPa is included in a total amount of 0.02 to 98 wt %, preferably 2 to 85 wt %, preferably 4 to 75 wt %, further preferably 6 to 70 wt %, still further preferably 8 to 60 wt %, in particular, 8 to 20 wt %, relative to the total weight of the antiperspirant cosmetic agent.

As a component a) of the cosmetic agents according to the present invention, at least one odorant may also be included. Preferably, however, mixtures of different odorants that together produce an appealing odorous note are used. Odorants that can be used within the framework of the present invention are, for example, disclosed in document DE 10 2010 063 250 A1.

Especially pleasant-smelling antiperspirant cosmetic agents according to the present invention are obtained when the at least one odorant is included in a total amount of 0.00001 to 15 wt %, preferably 0.001 to 9 wt %, preferably 0.01 to 8 wt %, further preferably 0.1 to 7 wt %, still further preferably 0.2 to 6 wt %, in particular, 0.2 to 2 wt %, relative to the total weight of the antiperspirant cosmetic agent.

The antiperspirant cosmetic agents according to the present invention may also include a wax as the component a). Preferably, this wax is selected from the group consisting of: (i) fatty acid glycerol mono-, di-, and triesters; (ii) Butyrospermum Parkii (shea butter); (iii) esters of saturated, monohydric C₈₋₁₈ alcohols with saturated C₁₂₋₁₈ monocarboxylic acids; (iv) linear primary C₁₂ to C₂₄ alkanols; (v) esters of a saturated, monohydric C₁₆₋₆₀ alkanol and a saturated C₈ to C₃₆ monocarboxylic acid; (vi) glycerol triesters of saturated linear C₁₂₋₃₀ carboxylic acids, which may be hydroxylated; (vii) natural plant-based waxes; (viii) animal waxes; (ix) synthetic waxes; and; (x) mixtures thereof. Waxes that can be preferably used within the framework of the present invention are disclosed in document DE 10 2012 222 692 A1.

Within the framework of the present invention, it is preferable when the wax is included in a total amount of 0.01 to 50 wt %, preferably 3 to 40 wt %, preferably 5 to 30 wt %, in particular, 6 to 25 wt %, relative to the total weight of the antiperspirant cosmetic agent.

According to an embodiment of the present invention, it may be provided that the antiperspirant cosmetic agents according to the present invention contain, as a component b), a propellant in a total amount of 0 to 99 wt % relative to the total weight of the antiperspirant cosmetic agent. When the cosmetic agents according to the present invention include a propellant, the propellant is preferably included in a total amount of 1 to 98 wt %, preferably 20 to 90 wt %, preferably 30 to 85 wt %, in particular, 40 to 75 wt %, relative to the total weight of the antiperspirant cosmetic agent. In this case, the cosmetic agents according to the present invention are prepared as propellant-driven aerosols. Preferred propellants (propellant gases) are propane, propene, n-butane, iso-butane, iso-butene, n-pentane, pentene, iso-pentane, iso-pentene, methane, ethane, dimethyl ether, nitrogen, air, oxygen, nitrogen oxide, laughing gas, 1,1,1,3 tetrafluoroethane, heptafluoro-n-propane, perfluoroethane, monochlorodifluoromethane, 1,1-difluoroethane, and tetrafluoropropenes, used individually or in combination. Hydrophilic propellant gases such as, for example, carbon dioxide can also be used advantageously for purposes of the present invention if the selected proportion of hydrophilic gases is low and an excess of lipophilic propellant gas (e.g., propane/butane) is present. Propane, n-butane, isobutane, and mixtures of these propellant gases are particularly preferred. It has been shown that the use of n-butane as a sole propellant gas can be particularly preferred according to the present invention.

As a third component c), the antiperspirant cosmetic agent according to the present invention includes at least one specific protein from Malvaceae of the genus Adansonia. The genus with the Latin designation Adansonia refers to deciduous trees in the form of baobab trees. Proteins that can be isolated from the African baobab tree are especially preferably used according to the present invention. Proteins that are isolated from the seeds of these trees have proven especially advantageous.

The at least one specific protein achieves an especially effective reduction of underarm sweat within the framework of the present invention when the at least one protein is included in a total amount of 0.5 to 60 wt %, preferably 1.0 to 50 wt %, preferably 1.5 to 40 wt %, further preferably 2.0 to 30 wt %, in particular, 2.0 to 20 wt % relative to the total weight of the antiperspirant cosmetic agent. Though no limitation to this theory is intended, use of the aforementioned amounts of the at least one specific protein results in a significant influence on the sweat gland(s) through gel formation of the protein in the ducts of the sweat glands, or through an influence on the charge balance within the sweat gland(s). This ensures an excellent antiperspirant action. Furthermore, the use of the aforementioned amounts of the at least one specific protein does not lead to unstable formulations, so that the stability of the antiperspirant cosmetic agents according to the present invention is ensured even over long storage periods.

Especially favorable results with regard to decreasing and/or reducing underarm wetness and with regard to the skin compatibility and storage stability are obtained when the at least one protein has an average molecular weight M_{w} of 150 to 100,000 Da, preferably 180 to 50,000 Da, preferably 200 to 10,000 Da, further preferably 250 to 8,000 Da, in particular, 300 to 5,000 Da. The average molecular weight M_{w} can be determined, for example, by gel permeation chromatography (GPC) (Andrews P.; “Estimation of the Molecular Weights of Proteins by Sephadex Gel-Filtration”; Biochem. J., 1964, 91, pp. 222-233).

According to a preferred embodiment of the present invention, the at least one protein has an isoelectric point that lies in the range of pH 4.0 to pH 10.0, preferably pH 4.0 to pH 9.5, in particular, pH 4.0 to pH 8.0. In particular, proteins that have an isoelectric point in the aforementioned pH range have proven advantageous within the framework of the present invention with respect to the antiperspirant action and the stability of the cosmetic agents according to the present invention.

An especially high antiperspirant action, skin compatibility, and storage stability are ensured within the framework of the present invention when the at least one protein brings about a change in the light absorption where there is a change in pH value of at least 0.5 in a pH range of pH 4.5 to pH 7.5, in particular, pH 5.0 to pH 7.0, with a concentration of 0.001 to 10 wt % protein relative to the total weight of the sample mixture used to measure the pH and a temperature of 20° C. Though no limitation to this theory is intended, the use of the at least one specific protein that brings about a change in the light absorption within a certain pH range results in a significantly elevated influence on the sweat gland(s) through pH-selective gel formation in the ducts of the sweat gland(s) or through disruption of the charge balance of the sweat gland(s). This ensures an excellent antiperspirant action of the cosmetic agents according to the present invention, which is comparable to the antiperspirant action of aluminum salt-containing or aluminum/zirconium salt-containing cosmetic agents of the prior art.

Within the framework of the present invention, it is preferable when the change in pH value is achieved by adding hydrogen carbonates or carbonates, in particular, sodium hydrogen carbonates.

According to a preferred embodiment of the present invention, the at least one protein is selected from the group consisting of: (i) non-modified proteins; (ii) hydrolyzed proteins; (iii) chemically modified proteins, in particular, hydrophobically and/or cationically and/or anionically modified proteins; (iv) physically modified proteins, in particular, fractionated and/or purified and/or irradiated proteins; (v) hydrolyzed non-modified proteins (vi) hydrolyzed and chemically modified proteins, in particular, hydrolyzed and hydrophobically and/or cationically and/or anionically modified proteins; (vii) hydrolyzed and physically modified proteins, in particular, fractionated and/or purified and/or irradiated proteins; and (viii) mixtures thereof.

The term “non-modified protein” is understood according to the present invention to mean proteins that have been treated neither by means of chemical processes—e.g., hydrolysis or chemical modification—nor by means of physical methods—e.g., purification, separation, or irradiation.

The term “hydrolyzed protein” or “protein hydrolysate” is understood according to the present invention to mean proteins that have been produced by chemical—in particular, alkaline or acid—hydrolysis, by enzymatic hydrolysis, and/or via a combination of both types of hydrolysis. All hydrolytically active enzymes, e.g., proteases, are suitable for the enzymatic degradation. Overviews of the production of protein hydrolysates can be found, for example, from G. Schuster and A. Domsch in Soaps, Oils, Fats, Waxes 108 (1982) 177 or Cosm. Toil. 99 (1984) 63, from H. W. Steisslinger in Parf. Kosm. 72 (1991) 556, and from F. Aurich et al. in Tens. Surf Det. 29 (1992) 389. Within the framework of the present invention, mixtures of individual amino acids obtained only by mixing the pure substances of the amino acids or total hydrolysates composed solely of individual amino acids do not fall under the terms “hydrolyzed protein” or “protein hydrolysate.”

The term “chemically modified protein” is understood within the framework of the present invention to mean a protein obtained by chemically reacting the reactive groups of the protein—in particular, the hydroxy, amine, imidazole, guanidino, and/or thiol groups of the side chains of the amino acids of the protein—with hydrophobic and/or cationic and/or anionic compounds.

The term “physically modified protein” within the meaning of the present invention is understood to mean proteins that have been modified by physical action, in particular, by heating and/or light and/or fractionation.

Within the framework of this embodiment, it is especially preferable when the at least one protein is selected from the group consisting of chemically modified, in particular, hydrophobically modified proteins. In this context, the hydrophobically modified protein has one or more C₄₋₃₀ carbon chains, wherein the C₄₋₃₀ hydrocarbon chains may be linear, cyclic, branched, unbranched, saturated, unsaturated, and aromatic, and wherein the C₄₋₃₀ hydrocarbon chains are bonded to the protein moiety via ether and/or ester and/or amine and/or amide bonds.

Moreover, within the framework of this embodiment, it is especially preferable when the at least one protein is selected from the group consisting of chemically modified, in particular, cationically modified proteins. Preferably, therefore, the cationically modified protein includes one or more moieties of the formula R¹—N⁺(CH3)₂—CH₂—CH(OH)—CH₂—X—R, in which R designates an alkyl group having 1 to 30 carbon atoms, an alkenyl group having 1 to 30 carbon atoms, a hydroxyalkyl group having 1 to 30 carbon atoms, in particular, a methyl group, a C₁₀₋₁₄ alkyl or C₁₀₋₁₄ alkenyl group, X designates an O, N, or S, and R designates the protein moiety. The proteins with the aforementioned moieties can be cationized by reacting the proteins with the corresponding halides of the above formula, wherein the aforementioned moieties can be bound to the protein via ether and/or ester and/or amide and/or amine bonds. The term “protein moiety” is understood within the framework of the present invention to mean the backbone of the corresponding protein which is formed by the linking of amino acids and to which the cationic group is bound via the aforementioned bonds.

Especially favorable results are obtained within the frame of this embodiment when the at least one protein is selected from the group consisting of hydrolyzed proteins.

In this context, it has proven advantageous when the at least one protein has a calcium content of 300 to 350 mg, a phosphorus content of 1,500 to 1,700 mg, a sulfur content of 410 to 450 mg, each relative to 1 kg of the protein, and a potassium content of 0.3 to 0.5 wt % relative to the total weight of the protein. The previously-specified contents can be determined, for example, by means of atomic emission spectrometry (ICP-OES) after microwave digestion with nitric acid (A. Oliveira et al.; “Evaluation of Metal Ions in Rice Samples: Extraction and Direct Determination by ICP-OES”; J. Braz. Chem. Soc, 2012, 23, pp. 838-845).

According to another especially preferred embodiment of the present invention, the at least one protein brings about a change in the light absorption of 1.5 to 90%, preferably 2 to 80%, preferably 2.5 to 70%, further preferably 3 to 65%, in particular, 3.5 to 60%. In particular, proteins from Malvaceae of the genus Adansonia that bring about the aforementioned change in the light absorption lead to an excellent antiperspirant action within the framework of the present invention. The change in the light absorption may occur at a change in the light transmittance of the sample mixture, in particular, by turbidity, as well as at the absorption of light through the sample mixture, in particular, through the protein itself.

The changes in the light absorption when there is a change in pH value of at least 0.5 such as are the basis of the present invention can be determined by measuring the light transmission of a beam of light through the sample mixture. The measurements of light transmission are performed with the use of a Metrohm Optrode 6.1115.000 at a wavelength of 574 nm (green-yellow) in mV (resolution 0.1 mV) in an open sample vessel at 23° C. and 1,013 mbar. The change in pH value within the pH range of 40 to 8.0 is achieved by slowly and continuously adding carbonate or hydrogen carbonate solution, preferably a 1-wt % sodium hydrogen carbonate solution, to the sample mixture while measuring the pH value with a pH electrode and stirring at a rate of 750 to 850 rpm. The change in the light absorption brought about by the at least one protein is calculated according to the formula ΔL=[(|Lᵢ|/L₀|)*100. In this formula, Lᵢ denotes the light transmission before and after a change in the pH value of at least 0.5 in the pH range of 4.0 to 8.0, preferably 4.5 and 7.5, in particular, pH 5.0 and 7.0, i.e., for example, light transmission at pH 5.0 minus light transmission at pH 6.0. In this formula, L₀ denotes the differential of the light transmission at pH 4.0 and at pH 8.0, preferably at pH 4.5 and at pH 7.5, in particular, at pH 5.0 and at pH 7.0, i.e., for example, light transmission at pH 8.0 minus light transmission at pH 4.0. The at least one specific protein in the antiperspirant cosmetic agents according to the present invention brings about a change in the light absorption of 1 to 100% as determined according to a method above. The present invention is not, however, limited to antiperspirant cosmetic compositions including at least one specific protein that brings about a change in the light absorption of 1 to 100% as determined by one of the above methods. The present invention also encompasses antiperspirant cosmetic compositions including at least one specific protein that brings about a change in the light absorption of 1 to 100% according to other methods. Within the framework of the present invention, it is preferable when the concentration of the at least one protein in the mixture used to determine the change in the light absorption is 0.005 to 10 wt %, preferably 0.05 to 5 wt %, preferably 0.07 to 3 wt %, in particular, 0.09 to 2 wt % relative to the total weight of the sample mixture used to determine the change in the light absorption.

Preferably according to the present invention, the at least one protein brings about a change in the light absorption when there is a change in pH value of at least 0.5 and at most 3.5, preferably at least 0.5 and at most 2.5, in particular, at least 0.5 and at most 1.5. The change in the pH value can be achieved, in particular, by adding acids or bases, preferably bases in the form of carbonates or hydrogen carbonates, in the appropriate amount.

According to another preferred embodiment of the present invention, the antiperspirant cosmetic agent has a pH value of pH 2 to pH 10. Within this range, the cosmetic agents according to the present invention can be stably formulated without encountering unwanted interactions between the ingredients of the antiperspirant cosmetic agents according to the present invention. The desired pH value can be adjusted according to the present invention by using acids and bases known to a person skilled in the art and commonly used in antiperspirant cosmetic agents.

It is furthermore preferable according to the present invention when the antiperspirant cosmetic agent additionally includes at least one preservative. Preservatives preferred according to the present invention are formaldehyde releasers, iodopropynyl butylcarbamates, parabens, phenoxyethanol, ethanol, benzoic acid and salts thereof, dibromodicyanobutane, 2-bromo-2-nitropropane-1,3-diol, imidazolidinyl urea, 5-chloro-2-methyl-4-isothiazoline-3-one, 2-chloroacetamide, benzalkonium chloride, benzyl alcohol, salicylic acid, and salicylates. Further preservatives that can be used within the framework of the present invention are the substances listed in Annex 6 of the Cosmetics Regulation, and cosmetic raw materials having preservative properties or raw materials that support or strengthen the preservative action of the aforementioned preservatives. The preservatives are preferably included in a total amount of 0.01 to 10 wt %, preferably 0.1 to 7 wt %, preferably 0.2 to 5 wt %, in particular, 0.3 to 2.0 wt %, relative to the total weight of the antiperspirant cosmetic agent.

Within the framework of the present invention, it is preferable when the antiperspirant cosmetic agent is present as a water-in-oil emulsion. This may entail, in particular, a sprayable water-in-oil emulsion that can be sprayed by means of a propellant. In this context, it is preferable when the antiperspirant cosmetic agent according to the present invention existing in the form of a water-in-oil emulsion includes the at least one protein in a total amount of 0.1 to 70 wt %, preferably 0.5 to 60 wt %, preferably 1.0 to 50 wt %, further preferably 1.5 to 40 wt %, still further preferably 2.0 to 30 wt %, in particular, 2.0 to 20 wt % relative to the total weight of the antiperspirant cosmetic agent.

It may also, however, be preferable according to the present invention when the antiperspirant cosmetic agent exists as an oil-in-water emulsion. In this case, the cosmetic agent according to the present invention is preferably sprayed as a propellant-free pump spray or squeeze spray, or applied as a roll-on. In this context, it is preferable when the antiperspirant cosmetic agent existing in the form of an oil-in-water emulsion includes the at least one protein in a total amount of 0.1 to 70 wt %, preferably 0.5 to 60 wt %, preferably 1.0 to 50 wt %, further preferably 1.5 to 40 wt %, still further preferably 2.0 to 30 wt %, in particular, 2.0 to 20 wt % relative to the total weight of the antiperspirant cosmetic agent.

According to another preferred embodiment of the present invention, the cosmetic agents according to the present invention may include only a low content of free water, or no free water. Free water within the meaning of the present invention is understood to mean water that is different from crystal water, hydration water, or similarly molecularly-bound water of the components used. The antiperspirant cosmetic agent preferably includes free water in a total amount of less than 10 wt %, preferably less than 8 wt %, preferably at least 5 wt %, further preferably less than 3 wt %, still more preferably less than 1 wt %, in particular, 0 wt % relative to the total weight of the antiperspirant cosmetic agent.

In the framework of another embodiment, however, it is also preferable according to the present invention when the antiperspirant cosmetic agent is present as an aqueous, aqueous-alcoholic, or aqueous-glycolic solution. Because the cosmetic agents according to the present invention include no antiperspirant aluminum and/or zirconium halides and/or hydroxy halides, the antiperspirant action of which is diminished by the addition of protic solvents, protic solvents such as aqueous solutions may, according to the present invention, be used to formulate the antiperspirant cosmetic agents according to the present invention, without encountering a significant reduction of the antiperspirant action. Adding the at least one specific protein therefore ensures an effective influence on the sweat gland(s) and thus an excellent antiperspirant action even with the use of protic solvents.

In the context of this embodiment of the present invention, it has surprisingly been determined that the influence of the at least one specific protein on the sweat gland(s) can be significantly increased when the antiperspirant cosmetic agents according to the present invention include free water in an amount of 5 to 99 wt % relative to the total weight of the antiperspirant cosmetic agent. In an especially preferred embodiment of the present invention, the antiperspirant cosmetic agent therefore includes free water in a total amount of 5 to 96 wt %, preferably 15 to 80 wt %, preferably 30 to 70 wt %, in particular, 40 to 60 wt %, relative to the total weight of the antiperspirant cosmetic agent.

It is furthermore preferred in the context of this embodiment when the antiperspirant cosmetic agent includes ethanol in a total amount of 1 to 99 wt %, preferably 5 to 70 wt %, preferably 7 to 50 wt %, in particular, 10 to 30 wt %, relative to the total weight of the antiperspirant cosmetic agent. As stated previously, using the at least one specific protein makes it possible to use large amounts of protic solvents, such as ethanol, without adversely affecting the antiperspirant action of the antiperspirant cosmetic agent according to the present invention.

The antiperspirant cosmetic agent according to the present invention can be applied by means of different methods. According to a preferred embodiment, the antiperspirant cosmetic agent is prepared as a spray application. The spray application is performed with a spray device having a container containing a filling made of the liquid, viscous and flowable, suspended, or powdered antiperspirant cosmetic agent according to the present invention. The filling may be under the pressure of a propellant (pressure gas can, pressure gas packaging, aerosol packaging), or may entail a pump sprayer that is used mechanically, without propellant gas (pump spray/squeeze bottle). The antiperspirant cosmetic agent can be sprayed physically, mechanically, or electromechanically, for example, by the piezoelectric effect or electric pumps. Containers and sampling devices that can be used within the framework of this embodiment are, for example, disclosed in publication DE 10 2012 222 692 A1.

The antiperspirant cosmetic agent may furthermore preferably be prepared as a stick, a soft solid, a cream, a gel, a roll-on, or a loose or compact powder. The formulation of the antiperspirant cosmetic agents according to the present invention in a certain administration form, for example, an antiperspirant roll-on, an antiperspirant stick, or an antiperspirant gel, preferably depends on the requirements of the intended use. Depending on the intended use, the antiperspirant cosmetic agents according to the present invention may therefore be present in solid, semisolid, liquid, dispersed, emulsified, suspended, gel, multiphase, or powdered form. Within the meaning of the present invention, the term “liquid” also includes any types of dispersions of solids in liquids. Furthermore, within the meaning of the present invention, multiphase antiperspirant cosmetic agents according to the present invention are understood to mean agents which have at least two different phases with a phase separation, and in which the phases may be situated horizontally, i.e., one above the other, or vertically, i.e., next to one another. The application may take place with a roller ball applicator, for example, or by means of a solid stick.

However, it may also be preferred within the framework of the present invention for the antiperspirant cosmetic agent to be included on and/or in a disposable substrate selected from the group of wipes, pads, and daubs. Particularly preferred are wet wipes, i.e., wet wipes which are prepackaged, preferably individually packaged, for the user, such as are well known, for example, from the area of glass cleaning or from the area of wet toilet wipes. Such wet wipes, which advantageously may also include preservatives, are impregnated or loaded with an antiperspirant cosmetic agent according to the present invention and preferably individually packaged. Preferred substrate materials are selected from porous flat wipes. These wipes include wipes made of woven and nonwoven (fleece) synthetic and natural fibers, felt, paper, or foam, such as hydrophilic polyurethane foam. According to the present invention, preferred deodorizing or antiperspirant substrates may be obtained by soaking or impregnating, or also fusing, an antiperspirant cosmetic agent according to the present invention on a substrate.

Preferably according to the present invention, the antiperspirant cosmetic agent includes at least one further auxiliary substance selected from the group consisting of (i) emulsifiers and/or surfactants, (ii) thickening agents, (iii) chelating agents, (iv) deodorant active ingredients, (v) monohydric and/or polyhydric alcohols and/or polyethylene glycols, (vi) skin-cooling active ingredients, (vii) pH adjusting agents, (viii) skin care active ingredients such as moistures, skin-soothing substances, skin-lightening substances, and skin-smoothing substances, and (ix) mixtures thereof.

Emulsifiers and surfactants which are preferably suitable according to the present invention are selected from anionic, cationic, nonionic, amphoteric, in particular ampholytic and zwitterionic, emulsifiers and surfactants. Surfactants are amphiphilic (bifunctional) compounds composed of at least one hydrophobic moiety and at least one hydrophilic moiety. The hydrophobic moiety is preferably a hydrocarbon chain including 8 to 28 carbon atoms, which may be saturated or unsaturated, linear or branched. This C₈ to C₂₈ alkyl chain is particularly preferably linear. Emulsifiers and surfactants that can preferably be used within the framework of the present invention are disclosed, for example, in documents DE 10 2012 222 692 A1, DE 10 2010 063 250 A1, and DE 10 2010 055 816 A1.

For thickening the antiperspirant cosmetic agents according to the present invention, it is preferable to use substances selected from cellulose ethers, xanthan gum, sclerotium gum, succinoglycans, polygalactomannans, pectins, agar, carrageenan, tragacanth, gum arabic, karaya gum, tara gum, gellan, gelatins, propylene glycol alginate, alginic acids and the salts thereof, polyvinylpyrrolidones, polyvinyl alcohols, polyacrylamides, physically modified starches (by pregelatinization, for example) and/or chemically modified starches, acrylic acid-acrylate copolymers, acrylic acid-acrylamide copolymers, acrylic acid-vinylpyrrolidone copolymers, acrylic acid-vinyl formamide copolymers, and polyacrylates. Especially preferred thickening agents are furthermore selected from carbomers. Carbomers are thickening crosslinked polymers of acrylic acid, methacrylic acid, and salts thereof. The crosslinking can be performed by means of polyfunctional compounds such as polyalkylene ethers of polysaccharides or polyalcohols, e.g., sucrose allyl ethers, pentaerythritol allyl ethers, or propylene allyl ethers. Homopolymers of acrylic acid or salts thereof that are crosslinked with a pentaerythritol allyl ether, a sucrose allyl ether, or a propylene allyl ether are preferred within the framework of the present invention. One thickening agent that can be used within the framework of the present invention is a copolymer of C₁₀₋₃₀ alkyl acrylate, acrylic acid, methacrylic acid and esters thereof that is crosslinked with a sucrose allyl ether or a pentaerythritol allyl ether. Carbomer-based thickening agents are products available under the trade name Carbopol® (BF Goodrich, Ohio, USA), e.g., Carbopol 934, Carbopol 940, Carbopol 941, Carbopol 971, Carbopol 974, Carbopol EZ2, Carbopol ETD 2001, Carbopol ETD 2020, Carbopol ETD 2050, Carbopol Ultrez 10, Carbopol Ultrez 20, or Carbopol Ultrez 21.

Lipophilic thickening agents can also be used to thicken the antiperspirant cosmetic agents according to the present invention. Lipophilic thickening agents that are preferable according to the present invention are selected from hydrophobized clay minerals, bentonites, pyrogenic silicic acids, and derivatives thereof.

In order to further support the influencing of the sweat gland(s) by the at least one specific protein, it may be advantageous to add—to the antiperspirant cosmetic agents according to the present invention—at least one chelating agent in a total amount of 0.01 to 3.0 wt %, preferably 0.02 to 1.0 wt %, in particular, 0.05 to 0.1 wt % relative to the total weight of the antiperspirant agent according to the present invention. Within the framework of the present invention, preferred chelating agents are selected from the group consisting of: β-alaninediacetic acid; cyclodextrin; diethylenetriaminepentamethylenephosphonic acid; sodium, potassium, calcium disodium, ammonium and triethanolamine salts of ethylenediaminetetraacetic acid (EDTA); etidronic acid; hydroxyethyl ethylenediamine triacetic acid (HEDTA) and sodium salts thereof; sodium salts of nitrilotriacetic acid (NTA); diethylenetriaminepentaacetic acid; phytic acid; hydroxypropyl cyclodextrin; methylcyclodextrin; aminotrimethylene phosphonate pentasodium; ethylenediamine tetramethylene phosphonate pentasodium; diethylenetriamine pentaacetate pentasodium; pentasodium triphosphate; potassium EDTMP; sodium EDTMP; sodium dihydroxyethyl glycinate; sodium phytate; sodium polydimethylglycinophenolsulfonate; tetrahydroxyethyl ethylenediamine; tetrahydroxyethyl ethylene diamine; tetrahydroxypropyl ethylene diamine; tetrapotassium etidronate; tetrasodium etidronate; tetrasodium iminodisuccinate; trisodium ethylenediamine disuccinate; tetrasodium-N,N-bis(carboxymethyl) glutamate; tetrasodium-DL-alanine-N,N-diacetate; and deferoxamine.

The deodorizing action of the antiperspirant cosmetic agents according to the present invention can be further increased when at least one deodorant active ingredient having an antibacterial and/or bacteriostatic and/or enzyme-inhibiting and/or odor-neutralizing and/or odor-absorbing action is included in a total amount of 0.0001 to 40 wt %, preferably 0.2 to 20 wt %, preferably 1 to 15 wt %, in particular, 1.5 to 5 wt % relative to the total weight of the antiperspirant cosmetic agent according to the present invention. If ethanol is used in the agents according to the present invention, it is not considered within the framework of the present invention to be a deodorant active ingredient, but rather a component of the carrier. Deodorant active ingredients according to the present invention are disclosed, for example, in document DE 10 2010 063 250 A1.

Preferred antiperspirant cosmetic agents according to the present invention further include at least one water-soluble polyhydric C₂₋₉ alkanol having 2 to 6 hydroxy groups and/or at least one water-soluble polyethylene glycol having 3 to 50 ethylene oxide units, and mixtures thereof. This does not include the aforementioned deodorant active ingredients in the form of 1,2-alkanediols. Preferred alkanols and water-soluble polyethylene glycols are described, for example, in document DE 10 2010 063 250 A1.

According to another embodiment of the present invention, the antiperspirant cosmetic agents further include at least one skin-cooling active ingredient. Skin-cooling active ingredients that are suitable according to the present invention are, for example, menthol, isopulegol as well menthol derivatives, e.g. menthyl lactate, menthyl glycolate, menthyl ethyl oxamate, menthyl pyrrolidone carboxylic acid, menthyl methyl ether, menthoxypropanediol, menthone glycerin acetal (9-methyl-6-(1-methylethyl)-1,4-dioxaspiro(4,5)decane-2-methanol), monomenthyl succinate, 2-hydroxymethyl-3,5,5-trimethylcyclohexanol, and 5-methyl-2-(1-methylethyl)cyclohexyl-N-ethyloxamate. Preferred skin-cooling active ingredients are menthol, isopulegol, menthyl lactate, menthoxypropanediol, menthyl pyrrolidone carboxylic acid, and 5-methyl-2-(1-methylethyl)cyclohexyl-N-ethyloxamate, and mixtures of these substances, in particular, mixtures of: menthol and menthyl lactate; menthol, menthol glycolate, and methyl lactate; menthol and menthoxypropanediol; or menthol and isopulegol.

Acids and/or alkalizing agents and/or buffers are preferably used as pH adjusting agents according to the present invention. Acids preferably used according to the present invention are inorganic acids (e.g., hydrochloric acid, sulfuric acid, or phosphoric acid) or organic acids (e.g., citric acid, tartaric acid, or malic acid). The alkalizing agents that can be used according to the present invention are preferably selected from the group consisting of ammonia, basic amino acids, alkali hydroxides, carbonates, hydrogen carbonates, alkanol amines, e.g., amino-2-methyl-1-propanol, monoethanolamine, triethanolamine, diethanolamine, and triisopropanolamine, alkali metal metasilicates, urea, morpholine, N-methylglucamine, imidazole, alkali phosphates, and alkali hydrogen phosphates. Lithium, sodium, and potassium—in particular, sodium or potassium—are preferably used as alkali metal ions. Particularly suitable buffer systems within the framework of the present invention are carbonic acid-bicarbonate buffer, carbonic acid-silicate buffer, acetic acid-acetate buffer, phosphate buffer, ammonia buffer, citric acid or citrate buffer, tris(hydroxymethyl)-aminomethane-based buffer, 4-(2-hydroxyethyl)-1-piperazine ethanesulfonic acid-based buffer, 4-(2-hydroxyethyl)-piperazine-1-propanesulfonic acid-based buffer, 2-(N-morpholino)ethanesulfonic acid-based buffer, and barbital-acetate buffer. The selection of the appropriate buffer system depends here on the desired pH value of the antiperspirant cosmetic agents according to the present invention.

The preferred embodiments described hereinbelow include no antiperspirant aluminum and/or zirconium halides and/or hydroxy halides:

In a preferred embodiment, the antiperspirant cosmetic agents according to the present invention are characterized by including, relative to the total weight of the antiperspirant cosmetic agent:
at least one protein in a total amount of 0.5 to 60 wt %, preferably 1.0 to 50 wt %, preferably 1.5 to 40 wt %, further preferably 2.0 to 30 wt %, in particular, 2.0 to 20 wt %;
12 to 98 wt %, preferably 25 to 55 wt %, preferably 30 to 50 wt %, in particular, 35 to 45 wt % water;
at least one emulsifier and/or one surfactant;
at least one pH adjusting agent;
at least one preservative; and
at least one substance selected from the group consisting of cosmetic oils that are liquid at 20° C. and 1,013 hPa, odorants, and waxes,
   wherein the at least one protein is found in Malvaceae of the genus Adansonia, and wherein the at least one protein brings about a change in the light absorption of 1 to 100% when there is a change in pH value of at least 0.5 in a pH range of pH 4.0 to pH 8.0, a temperature of 20° C. to 40°, and a concentration of the protein of 0.001 to 10 wt % relative to the total weight of the sample mixture used to determine the change in the light absorption.

In another preferred embodiment, the antiperspirant cosmetic agents according to the present invention are characterized by including, relative to the total weight of the antiperspirant cosmetic agent:
at least one protein in a total amount of 0.5 to 60 wt %, preferably 1.0 to 50 wt %, preferably 1.5 to 40 wt %, further preferably 2.0 to 30 wt %, in particular, 2.0 to 20 wt %;
12 to 98 wt %, preferably 25 to 55 wt %, preferably 30 to 50 wt %, in particular, 35 to 45 wt % water;
at least one emulsifier and/or one surfactant;
at least one pH adjusting agent;
at least one preservative;
0.01 to 2 wt %, preferably 0.1 to 1 wt %, preferably 0.2 to 0.7 wt %, in particular, 0.3 to 0.5 wt % a thickening agent; and
at least one substance selected from the group consisting of cosmetic oils that are liquid at 20° C. and 1,013 hPa, odorants, and waxes,
   wherein the at least one protein is found in Malvaceae of the genus Adansonia, and wherein the at least one protein brings about a change in the light absorption of 1 to 100% when there is a change in pH value of at least 0.5 in a pH range of pH 4.0 to pH 8.0, a temperature of 20° C. to 40°, and a concentration of the protein of 0.001 to 10 wt % relative to the total weight of the sample mixture used to determine the change in the light absorption.

In a preferred embodiment, the antiperspirant cosmetic agents according to the present invention are characterized by including, relative to the total weight of the antiperspirant cosmetic agent:
at least one protein in a total amount of 0.5 to 60 wt %, preferably 1.0 to 50 wt %, preferably 1.5 to 40 wt %, further preferably 2.0 to 30 wt %, in particular, 2.0 to 20 wt %;
12 to 98 wt %, preferably 25 to 55 wt %, preferably 30 to 50 wt %, in particular, 35 to 45 wt % water;
at least one propellant in a total amount of 1 to 98 wt %, preferably 20 to 90 wt %, preferably 30 to 85%, in particular, 40 to 75 wt %;
at least one emulsifier and/or one surfactant;
at least one pH adjusting agent;
at least one preservative; and
at least one substance selected from the group consisting of cosmetic oils that are liquid at 20° C. and 1,013 hPa, odorants, and waxes,
   wherein the at least one protein is found in Malvaceae of the genus Adansonia, and wherein the at least one protein brings about a change in the light absorption of 1 to 100% when there is a change in pH value of at least 0.5 in a pH range of pH 4.0 to pH 8.0, a temperature of 20° C. to 40°, and a concentration of the protein of 0.001 to 10 wt % relative to the total weight of the sample mixture used to determine the change in the light absorption.

In another preferred embodiment, the antiperspirant cosmetic agents according to the present invention are characterized by including, relative to the total weight of the antiperspirant cosmetic agent:
at least one protein in a total amount of 0.5 to 60 wt %, preferably 1.0 to 50 wt %, preferably 1.5 to 40 wt %, further preferably 2.0 to 30 wt %, in particular, 2.0 to 20 wt %;
12 to 98 wt %, preferably 25 to 55 wt %, preferably 30 to 50 wt %, in particular, 35 to 45 wt % water;
at least one propellant in a total amount of 1 to 98 wt %, preferably 20 to 90 wt %, preferably 30 to 85%, in particular, 40 to 75 wt %;
at least one emulsifier and/or one surfactant;
at least one pH adjusting agent;
at least one preservative;
0.01 to 2 wt %, preferably 0.1 to 1 wt %, preferably 0.2 to 0.7 wt %, in particular, 0.3 to 0.5 wt % a thickening agent; and
at least one substance selected from the group consisting of cosmetic oils that are liquid at 20° C. and 1,013 hPa, odorants, and waxes,
   wherein the at least one protein is found in Malvaceae of the genus Adansonia, and wherein the at least one protein brings about a change in the light absorption of 1 to 100% when there is a change in pH value of at least 0.5 in a pH range of pH 4.0 to pH 8.0, a temperature of 20° C. to 40°, and a concentration of the protein of 0.001 to 10 wt % relative to the total weight of the sample mixture used to determine the change in the light absorption.

Within the framework of the present invention, it may be provided that the cosmetic agent according to the present invention is used in the framework of a two-component agent. For this purpose, the individual components are preferably stored in separated containers and applied to the skin either one after the other in any order, or at the same time.

Another subject matter of the present invention is therefore a packaging unit (kit-of-part) comprising—prepared separately from one another—
a) at least one first container (C1) containing a cosmetic agent (M1) comprising at least one antiperspirant active ingredient, and
b) at least one second container (C2) containing a cosmetic agent (M2) including at least one protein, wherein the at least one protein is found in Malvaceae of the genus Adansonia, wherein the at least one protein brings about a change in the light absorption of 1 to 100% when there is a change in pH value of at least 0.5 in a pH range of pH 4.0 to pH 8.0, a temperature of 20° C. to 40°, and a concentration of the protein of 0.001 to 10 wt % relative to the total weight of the sample mixture used to determine the change in the light absorption, and wherein the cosmetic agent includes no aluminum and/or zirconium halides and/or hydroxy halides.

The term “antiperspirant active ingredient” is understood according to the present invention to mean active ingredients that decrease or reduce the perspiration of the sweat glands of the body. This does not, however, include the proteins from Malvaceae of the genus Adansonia that are included in the cosmetic agent (M2) and bring about a change in the light absorption under the conditions described above.

What has been said regarding the cosmetic agents according to the present invention also applies mutatis mutandis with respect to the cosmetic agent (M2) in the container (C2).

Another subject matter of the present invention is the use of a protein to at least partially influence the sweat gland(s), wherein the at least one protein is found in Malvaceae of the genus Adansonia, and wherein the at least one protein brings about a change in the light absorption of 1 to 100% when there is a change in pH value of at least 0.5 in a pH range of pH 4.0 to pH 8.0, a temperature of 20° C. to 40°, and a concentration of the protein of 0.001 to 10 wt % relative to the total weight of the sample mixture used to determine the change in the light absorption.

Influencing of the sweat gland(s) is understood according to the present invention to mean influencing the sweat gland(s) in such a manner as to decrease or reduce the secretion of sweat from the duct. Though no limitation to this theory is intended, this may occur, for example, through formation of a gel and/or precipitation of the at least one specific protein in the duct of the sweat gland or the ducts of the sweat glands. Furthermore, however, using the at least one specific protein also leads to a disruption of the charge balance within the ducts of the sweat glands. What has been said regarding the cosmetic antiperspirant agents according to the present invention also applies mutatis mutandis with respect to the use according to the present invention.

Yet another subject matter of the present invention is the use of a combination, containing
a) at least one substance selected from the group consisting of cosmetic oils that are liquid at 20° C. and 1,013 hPa, odorants, and waxes;
b) propellants in a total amount of 0 to 99 wt %, relative to the total weight of the antiperspirant cosmetic agent; and
c) at least one protein in a total amount of 0.1 to 70 wt % relative to the total weight of the antiperspirant cosmetic agent, wherein the at least one protein is found in Malvaceae of the genus Adansonia, wherein the at least one protein brings about a change in the light absorption of 1 to 100% when there is a change in pH value of at least 0.5 in a pH range of pH 4.0 to pH 8.0, a temperature of 20° C. to 40°, and a concentration of the protein of 0.001 to 10 wt % relative to the total weight of the sample mixture used to determine the change in the light absorption; and
   wherein the combination includes no aluminum and/or zirconium halides and/or hydroxy halides, to reduce and/or prevent sweat, in particular, underarm sweat or sweat from other regions of the body.

The term “combination in the meaning of the present invention encompasses a mixture of the above-cited ingredients a), b), and c). What has been said regarding the antiperspirant cosmetic agents according to the present invention and regarding the use according to the present invention also applies mutatis mutandis with respect to the use of the aforementioned combination.

Another subject matter of the present invention is an antiperspirant cosmetic agent containing:
a) at least one substance selected from the group consisting of cosmetic oils that are liquid at 20° C. and 1,013 hPa, odorants, and waxes;
b) propellants in a total amount of 0 to 99 wt %, relative to the total weight of the antiperspirant cosmetic agent; and
c) at least one protein isolated from Malvaceae of the genus Adansonia, preferably a protein isolated from the seeds of the genus Adansonia, in a total amount of 0.1 to 70 wt % relative to the total weight of the antiperspirant cosmetic agent, wherein the protein is hydrolyzed,
   wherein the antiperspirant cosmetic agents include no aluminum and/or zirconium halides and/or hydroxy halides.

Within the framework of this subject matter, it is especially preferable when the at least one protein has a calcium content of 300 to 350 mg, a phosphorus content of 1,500 to 1,700 mg, a sulfur content of 410 to 450 mg, each relative to 1 kg of the protein, and a potassium content of 0.3 to 0.5 wt % relative to the total weight of the protein. The previously-specified contents can be determined, for example, by means of atomic emission spectrometry (ICP-OES), as previously stated.

What has been said regarding the antiperspirant cosmetic agents according to the present invention and regarding the use according to the present invention also applies mutatis mutandis with respect to this subject matter of the present invention.

Finally, another subject matter of the present invention is a non-therapeutic cosmetic method for preventing and/or reducing body perspiration with which an antiperspirant cosmetic agent according to the present invention or a packaging unit according to the present invention is applied onto the skin, in particular, the skin of the armpits, and remains on the skin of the armpits for at least 1 hour, preferably for at least 2 hours, preferably for at least 4 hours, in particular, for at least 6 hours.

If, within the framework of the method according to the present invention, the packaging unit is used in accordance with the present invention, it may be provided that first the cosmetic agent (M1) in the container (C1) is applied, and then the cosmetic agent (M2) in the container (C2) is applied. It is, however, also possible to first apply the cosmetic agent (M2) in the container (C2), and then apply the cosmetic agent (M1) in the container (C1). Moreover, the cosmetic agent (M1) in the container (C1) and the cosmetic agent (M2) in the container (C2) may also be applied simultaneously. The amount of time between the use of the two agents (M1) and (M2) is 0 to 24 hours.

What has been said regarding the antiperspirant cosmetic agents according to the present invention, regarding the packaging unit according to the present invention, and regarding the use according to the present invention also applies mutatis mutandis with respect to this subject matter of the present invention.

The following examples illustrate the present invention, but do so in a non-limiting manner:

### Examples

1. Change in the light absorption

A hydrolyzed protein isolated from the seeds of the genus Adansonia was used.

The change in the light absorption brought about by the aforementioned proteins in a pH range of 4.0 to 8.0 with a change in pH value of at least 0.5 is determined as follows:

TABLE 1
Sample solution (amounts given in wt %)

| | E-I* |
|---|---|
| Hydrolyzed protein of the genus Adansonia^{a)} | 10 |
| HCl | to pH |
| water | to 100 |

*according to the present invention
^{a)}Baobab Tein NPNF; (INCI: Hydrolyzed Adansonia Digitata Seed Protein; 10-wt % aqueous solution; Neochem)

The change in the light absorption was determined by using a Metrohm Titrando 905 from Metrohm (USA), which is equipped with a Metrohm Optrode 6.1115.000 and a pH electrode from Metrohm. The Metrohm Titrando 905 is controlled via the software Tiamo from Metrohm. Next, 30 mL of a sample solution according to table 1, having a pH value of 3.0, was added into the open sample vessel of the Metrohm Titrando 905. Next, a 1-wt % sodium hydrogen carbonate solution was continuously added at 23° C. and 1,013 mbar with stirring (stirrer speed 8 of the Titrando 905, corresponding to about 750 to 850 rpm) up until a pH value of 7.5 was achieved. While the 1-wt % sodium hydrogen carbonate solution was being added, the light transmission of a beam of light through this sample solution was measured with a Metrohm Optrode 6.1115.000 at a wavelength of 574 nm (green-yellow) in mV (resolution 0.1 mV). Each measurement is performed twice, and the average value of the two is taken.

The change in the light absorption brought about by the aforementioned peptides was determined according to the formula ΔL=[(|Lᵢ|/|L₀|)*100. In this formula, Lᵢ denotes the light transmission before and after a change in the pH value of at least 0.5 in the pH range of 4.0 to 8.0, preferably 4.5 and 7.5, in particular, pH 5.0 and 7.0. In this formula, L₀ denotes the differential of the light transmission at pH 4.0 and at pH 8.0, preferably at pH 4.5 and at pH 7.5, in particular, at pH 5.0 and at pH 7.0.

With a change in pH value of 1.0 between pH 5.0 and 6.0 (differential of light absorption at pH 5.5 minus pH 6.0 forming the value Lᵢ) in a pH value range of 4.5 to 7.5 (differential of light absorption at pH 7.5 minus pH 4.5 forming the value L₀), this protein brought about a change in the light absorption AL of 40%.

2. In Vivo Testing of the Antiperspirant Effect

To determine the antiperspirant effect, an antiperspirant study was conducted on the backs of 16 female test subjects. The following antiperspirant agents were used for this purpose:

| Antiperspirant agent | Nr |
|---|---|
| Aqueous solution with 10% ACH, pH 4 | V-I |
| Aqueous solution with 2% protein^{a)}, pH 2-4 | E-II** |

* active substance
**according to the present invention
^{a)}Baobab Tein NPNF; (INCI: Hydrolyzed Adansonia Digitata Seed Protein; 10-wt % aqueous solution; Neochem)

40 μL of the antiperspirant agent V-I and 75 μL of the cosmetic agent E-II according to the present invention were applied to the backs of 16 subjects, one on each side of the backbone. After 5 minutes, the treated areas were covered with occlusive non-adsorptive film. After 2 hours, these non-adsorptive pads were removed. The compositions were applied to the backs of the test subjects in the aforementioned manner for four consecutive days. At 24 hours after the last application of the composition, absorbent pads were applied onto the backs of the test subjects at the places where the compositions had previously been applied. Pads serving as a control were also applied to the other side of the backbone at the same height. After the subjects had sweated for about 15 minutes at 80° C. in a sauna, the amount of sweat absorbed by the pads was determined gravimetrically, wherein each composition was compared with the respectively-corresponding untreated place on the back. The sweat reduction was determined from the gravimetric determination of the amount of sweat, wherein all determined values were statistically significant.

The sweat reduction of the respective composition as compared to an untreated place on the skin is given in the following table:

| Nr | Sweat reduction |
|---|---|
| V-I | 50% |
| E-II | 25% |

The use of the specific protein leads to a significant decrease in the sweat reduction and to a satisfactory antiperspirant action.

3. Formulations

The protein used in the following examples is preferably a hydrolyzed protein isolated from the seeds of the genus Adansonia.

Antiperspirant cosmetic agents according to the present invention with a pH of 2.5 to 10.0 (amounts given in wt %)

| | 1 | 2 | 3 | 4 | 5 | 6 | 7 |
|---|---|---|---|---|---|---|---|
| Isopropyl myristate | 0.50 | 0.10 | 0.50 | 1.0 | 2.0 | 3.0 | 5.0 |
| Protein | 0.50 | 2.0 | 3.0 | 5.0 | 7.0 | 10 | 20 |
| Eumulgin B3 ^{b)} | 3.0 | 3.0 | 3.0 | 4.0 | 4.0 | 4.0 | 5.0 |
| Perfume | 0.10 | 0.20 | 0.30 | 0.30 | 0.50 | 0.8 | 1.0 |
| Preservative | 0.50 | 0.50 | 0.50 | 0.80 | 0.80 | 1.5 | 2.0 |
| pH adjusting agent | to pH | to pH | to pH | to pH | to pH | to pH | to pH |
| water | to 100 | to 100 | to 100 | to 100 | to 100 | to 100 | to 100 |

^{b)} Eumulgin B3 (INCI: Ceteareth-30; BASF)

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antiperspirant cosmetic agent, comprising:
b) at least one substance selected from the group consisting of cosmetic oils that are liquid at 20° C. and 1,013 hPa, odorants, and waxes;
c) propellants in a total amount of 0 to 99 wt %, relative to the total weight of the antiperspirant cosmetic agent; and
d) at least one protein in a total amount of 0.1 to 70 wt % relative to the total weight of the antiperspirant cosmetic agent, wherein the at least one protein is found in Malvaceae of the genus Adansonia, and wherein the at least one protein brings about a change in the light absorption of 1 to 100% when there is a change in pH value of at least 0.5 in a pH range of pH 4.0 to pH 8.0, a temperature of 20° C. to 40°, and a concentration of the protein of 0.001 to 10 wt % relative to the total weight of the sample mixture used to determine the change in the light absorption;
wherein the antiperspirant cosmetic agents include no aluminum and/or zirconium halides and/or hydroxy halides.

2. The antiperspirant cosmetic agent according to claim 1, wherein the at least one protein is included in a total amount of 0.5 to 60 wt % relative to the total weight of the antiperspirant cosmetic agent.

3. The antiperspirant cosmetic agent according to claim 1, wherein the at least one protein is included in a total amount of 3 to 20 wt % relative to the total weight of the antiperspirant cosmetic agent.

4. The antiperspirant cosmetic agent according to claim 1, wherein the at least one protein has an average molecular weight M_{w} of 150 to 100,000 Da.

5. The antiperspirant cosmetic agent according to claim 1, wherein the at least one protein has an average molecular weight M_{w} of 300 to 5,000 Da.

6. The antiperspirant cosmetic agent according to claim 1, wherein the at least one protein brings about a change in the light absorption where there is a change in pH value of at least 0.5 in a pH range of pH 4.5 to pH 7.5, with a concentration of 0.001 to 10 wt % protein relative to the total weight of the sample mixture used to measure the pH and a temperature of 20° C.

7. The antiperspirant cosmetic agent according to claim 1, wherein the change in pH value occurs due to the addition of hydrogen carbonates or carbonates

8. The antiperspirant cosmetic agent according to claim 1, wherein the change in pH value occurs due to the addition of sodium hydrogen carbonates.

9. The antiperspirant cosmetic agent according to claim 1, wherein the at least one protein is selected from the group consisting of: (i) non-modified proteins; (ii) hydrolyzed proteins; (iii) chemically modified proteins; (iv) physically modified proteins; (v) hydrolyzed non-modified proteins (vi) hydrolyzed and chemically modified proteins; (vii) hydrolyzed and physically modified proteins; and (viii) mixtures thereof.

10. The antiperspirant cosmetic agent according to claim 1, wherein the at least one protein brings about a change in the light absorption of 1.5 to 90%.

11. The antiperspirant cosmetic agent according to claim 1, wherein the at least one protein brings about a change in the light absorption of 3.5 to 60%.

12. The antiperspirant cosmetic agent according to claim 1, wherein the concentration of the protein in the mixture used to determine the change in the light absorption is 0.005 to 10 wt % relative to the total weight of the mixture used to determine the change in the light absorption.

13. The antiperspirant cosmetic agent according to claim 1, wherein the concentration of the protein in the mixture used to determine the change in the light absorption is 0.09 to 2 wt % relative to the total weight of the mixture used to determine the change in the light absorption.

14. The antiperspirant cosmetic agent according to claim 1, wherein the at least one protein brings about a change in the light absorption when there is a change in pH value of at least 0.5 and at most 3.5,

15. A packaging unit (kit-of-parts) comprising—prepared separately from one another—
a) at least one first container (C1) containing a cosmetic agent (M1) comprising at least one antiperspirant active ingredient, and
b) at least one second container (C2) containing a cosmetic agent (M2) including at least one protein, wherein the at least one protein is found in Malvaceae of the genus Adansonia, wherein the at least one protein brings about a change in the light absorption of 1 to 100% when there is a change in pH value of at least 0.5 in a pH range of pH 4.0 to pH 8.0, a temperature of 20° C. to 40°, and a concentration of the protein of 0.001 to 10 wt % relative to the total weight of the sample mixture used to determine the change in the light absorption, and wherein the cosmetic agent (M2) includes no aluminum and/or zirconium halides and/or hydroxy halides.

16. An antiperspirant cosmetic agent, including:
a) at least one substance selected from the group consisting of cosmetic oils that are liquid at 20° C. and 1,013 hPa, odorants, and waxes;
b) propellants in a total amount of 0 to 99 wt %, relative to the total weight of the antiperspirant cosmetic agent; and
c) at least one protein isolated from Malvaceae of the genus Adansonia, in a total amount of 0.1 to 70 wt % relative to the total weight of the antiperspirant cosmetic agent, wherein the protein is hydrolyzed, wherein the antiperspirant cosmetic agent includes no aluminum and/or zirconium halides and/or hydroxy halides.

17. The antiperspirant cosmetic agent according to claim 16, characterized in that the at least one protein has a calcium content of 300 to 350 mg, a phosphorus content of 1,500 to 1,700 mg, a sulfur content of 410 to 450 mg, each relative to 1 kg of the protein, and a potassium content of 0.3 to 0.5 wt % relative to the total weight of the protein.

18. A non-therapeutic cosmetic method for preventing and/or reducing body perspiration with which the antiperspirant cosmetic agent according to claim 1 is applied onto the skin, in particular, the skin of the armpits, and remains on the skin of the armpits for at least 1 hour.
